(19) United States
(12) Patent Application Publication
(10) Pub. No.: US 2021/0401722 A1
(43) Pub. Date: Dec. 30, 2021

(54) COSMETIC COMPOSITIONS COMPRISING BIODEGRADABLE POLYMERS

(71) Applicant: L'ORÉAL, Paris (FR)

(72) Inventors: Hubert LAM, New Providence, NJ (US); Phi Hoang DOAN, Clark, NJ (US); Mariko HASEBE, West New York, NJ (US)

(73) Assignee: L'ORÉAL, Paris (FR)

(21) Appl. No.: 16/913,379

(22) Filed: Jun. 26, 2020

(51) Int. Cl. A61K 8/85; A61K 8/891; A61Q 19/00; A61Q 5/00; A61Q 1/12; A61K 8/02; A61K 8/06

(52) U.S. Cl. CPC A61K 8/85; A61K 8/891; A61Q 19/00; A61K 2800/10; A61Q 1/12; A61K 8/022; A61K 8/064; A61Q 5/00

(57) ABSTRACT

Disclosed is a cosmetic composition that contains at least one polyhydroxyalkanoate (PHA) in the form of particles having an average diameter (d50) from 0.1 nm to 50 μm. The PHA may comprise between 0.1% w/w and 30% w/w of the total cosmetic composition, and some embodiments may utilize between 1% w/w and 8% w/w. The composition may include a silicone external phase or may be in the form of a loose or packed powder. Various PHAs may be utilized, although advantageously, the PHA may have a chain length 1 and 8 carbons. In some embodiments, the PHA may be selected to have an oil absorption of between 60 to 150 grams of a desired oil per 100 grams of PHA, where the desired oil may be, e.g., sebum. Further, the composition may optionally include one or more pigments. In use, the composition may be applied to a user's skin or hair.

### FIELD OF THE INVENTION

The present invention relates to cosmetic compositions, and specifically to cosmetic compositions utilizing polyhydroxyalkanoates and either having a silicone external phase or being in the form of a loose or pressed powder.

### BACKGROUND

Cosmetic compositions are typically developed to allow a user to place particular desired components, such as colored pigments, antioxidants, skin protectants, and moisturizing agents, texture powders on the body, face, or hair, without the product's aesthetics making it an unpleasant experience.

Some of these compositions utilize various fillers to provide desirable characteristics to the composition. However, cosmetic fillers are generally not soluble in oil or water phases, so formulators often need to create emulsions to ensure the product is shelf-stable. In some instances, silicone is used as the external, or continuous, phase. Further, some cosmetic compositions contain powders and can be used as opacifier, bulking agent, oil absorber, matifying agent. Depending on the type of powder used and how it interacts with the remainder of the formulation, the properties of the cosmetic composition may vary, and may even be harmful to the skin, leaving it feeling dull, dry, and/or rough.

Further, many consumers want products that contain ingredients that can be viewed as environmentally friendly and not creating negative impact on the environment. Consumers look for finished products that do not contribute new carbon to the atmosphere considering the environmental impact of production, use and degradation. Yet many cosmetic powders are not biodegradable. For example, poly(methyl methacrylate) (PMMA) and Nylon-12 are standard ingredients that are commonly used in cosmetics a bulking agent or opacifying agent, to create a full and smooth appearance on the skin, reduce shine/gloss, and impart a desired feel to formulations. However, neither PMMA nor Nylon-12 are not very biodegradable, and the typical manufacturing processes for making those materials is not environmentally friendly.

Therefore, there is a need for a biodegradable, environmentally-friendly powder material that can be used in cosmetic compositions, and specifically in cosmetic compositions having silicone as the external phase or having a form of a loose or pressed powder.

### BRIEF SUMMARY

Disclosed is a cosmetic composition that contains between 0.1% w/w and 30% w/w of at least one polyhydroxyalkanoate (PHA) in the form of particles having an average diameter (d50) from 0.1 nm to 50 μm. The PHA may be present in the composition in an amount from 1% w/w to 8% w/w. The composition may include a silicone external phase or may be in the form of a loose or packed powder. Advantageously, the PHA has a chain length between 1 and 8 carbons and more particularly between 2 and 6 carbons, such as between 2 and 5 carbons, such as between 2 and 4 carbons, such as 4 carbons. In certain embodiments, the PHA is selected from the group consisting of: poly-3-hydroxybutyrate (PHB), poly-3-hydroxyvalerate (PHV), poly-3-hydroxyhexanoate (PHH), poly-3-hydroxyoctanoate (PHO), poly (3-hydroxybutyrate-co-3-hydroxyvalerate) (PHBV), poly (3-hydroxybutyrate-co-3-hydroxyexanoate)(PHBH), poly (3-hydroxybutyrate-co-4-hydroxybutyrate), poly (3-hydroxyoctanoate-co-3-hydroxyundecen-10-enoate) (PHOU), and poly(3-hydroxybutyrate-co-3-hydroxyvalerate-co-4-hydroxyvalerate) (PHBW).

The PHA may be selected to have an oil absorption of between 60 to 150 grams of a desired oil per 100 grams of PHA.

The composition may optionally include one or more pigments. In use, the composition may be applied to a user's skin or hair.

### DETAILED DESCRIPTION

As used herein, articles such as “a” and “an” when used in a claim, are understood to mean one or more of what is claimed or described.

As used herein, the expression “at least one” means one or more and thus includes individual components as well as mixtures/combinations.

All percentages listed are by weight unless otherwise noted.

As used herein, the term “about [a number]” is intended to include values rounded to the appropriate significant digit. Thus, “about 1” would be intended to include values between 0.5 and 1.5, whereas “about 1.0” would be intended to include values between 0.95 and 1.05.

As used herein, the terms “include”, “includes” and “including” are meant to be non-limiting.

As used herein, “substituted” means comprising at least one substituent. Non-limiting examples of substituents for substitution include atoms, such as oxygen atoms and nitrogen atoms, as well as functional groups, such as hydroxyl groups, ether groups, alkoxy groups, acyloxyalky groups, oxyalkylene groups, polyoxyalkylene groups, carboxylic acid groups, amine groups, acylamino groups, amide groups, halogen containing groups, ester groups, thiol groups, sulphonate groups, thiosulphate groups, siloxane groups, and polysiloxane groups. The substituent(s) may be further substituted.

The present invention is drawn to a cosmetic composition and specifically to cosmetic compositions utilizing polyhydroxyalkanoates and having a silicone external phase. The cosmetic composition may be a water-in-silicone emulsion. In certain embodiments, the total amount of silicones in the composition may be greater than 10% w/w. In certain embodiments, the total amount of silicones in the composition may be greater than 15% w/w. In certain embodiments, the amount of silicones in the composition may be between 10% w/w and 40% w/w. The cosmetic composition may contain one or more silicones, which may include, but is not limited to, dimethicone, phenyl trimethicone or volatile silicone oils.

In certain embodiments, the cosmetic composition is a skin care, hair care, or make-up composition.

The cosmetic composition generally includes at least one polyhydroxyalkanoate (PHA). PHAs are known to be biodegradable and can be produced via, e.g., environmentally friendly fermentation processes.

Surprisingly, PHAs can be substituted for more traditional powders (such as PMMA or Nylon) in various bases to provide equivalent viscosity modification, mattifying effects, and sebum transfer resistance.

In certain embodiment, the PHA may have a chain length between 1 and 8 carbons and more particularly between 2 and 6 carbons, such as between 2 and 5 carbons, such as between 2 and 4 carbons, such as 4 carbons. All ranges are inclusive of endpoints. In certain embodiments, the PHA may include, but is not limited to, poly-3-hydroxybutyrate (PHB), poly-3-hydroxyvalerate (PHV), poly-3-hydroxyhexanoate (PHH), poly-3-hydroxyoctanoate (PHO), poly (3-hydroxybutyrate-co-3-hydroxyvalerate) (PHBV), poly (3-hydroxybutyrate-co-3-hydroxyexanoate) (PHBH), poly (3-hydroxybutyrate-co-4-hydroxybutyrate), poly (3-hydroxyoctanoate-co-3 -hydroxyundecen-10-enoate) (PHOU), poly (3-hydroxybutyrate-co-3 -hydroxyvalerate-co-4-hydroxyvalerate) (PHBW), or a combination thereof. The PHA may be a part of a copolymer, or the copolymer may derived from two or more PHAs.

Preferrably, the PHA is PHB, such as PHB available from TianAn Biopolymer under the ENMAT™ brand.

In certain embodiments, the total amount of PHA present in the composition is greater than about 0.01% w/w. In certain embodiments, the total amount of PHA present in the composition is greater than about 0.1% w/w. In certain embodiments, the total amount of PHA present in the composition is greater than about 0.5% w/w. In certain embodiments, the total amount of PHA present in the composition is greater than about 1% w/w. In certain embodiments, the total amount of PHA present in the composition is greater than about 2% w/w.

In certain embodiments, the total amount of PHA present in the composition is less than about 50% w/w. In certain embodiments, the total amount of PHA present in the composition is less than about 30% w/w. In certain embodiments, the total amount of PHA present in the composition is less than about 15% w/w. In certain embodiments, the total amount of PHA present in the composition is less than about 8% w/w.

In preferred embodiments, the total amount of PHA present in the composition is between about 0.1% w/w and about 30% w/w. In more preferred embodiments, the total amount of PHA present in the composition is between about 1% w/w and about 8% w/w. In the context of the present invention and of the appended claims, by “average particle diameter” it is meant particularly according, unless otherwise indicated, the diameter d50 (median value), that is, the value of the diameter below which 50% by weight of the particle population is found (see “A Guidebook to Particle Size Analysis” published by Horiba Instruments Inc. 2016, available on https://www.horiba.corn/fileadmin/uploads/Scientific/eMag/PSA/Guidebook/pdf/PSA_Guidebook.pdf). It can be determined by laser diffraction technique, according to the ISO 13320:2008 standard or determined with TEM Transmission Electron Microscopy.

In certain embodiments, the PHA is in the form of particles having an average diameter (d50) less than 100 nm. In certain embodiments, the d50 is less than 50 μm. In certain embodiments, the d50 is less than 30 μm. In certain embodiments, the d50 is less than 20 μm. In certain embodiments, the d50 is greater than 0.1 nm. In certain embodiments, the d50 is greater than 1 nm. In certain embodiments, the d50 is greater than 100 nm. In certain embodiments, the d50 is greater than 1 μm. In preferred embodiments, the d50 is between 0.1 nm and 50 μm, and more preferably between 1 μm and 20 μm.

In certain embodiments, the PHA is selected to have an oil absorption of between 60 to 150 grams of one or more desired oils per 100 grams of PHA. In certain embodiments, the desired one or more oils is natural or synthetic sebum. In certain embodiments, the desired one or more oils is a cosmetic oil present in the composition, such as a silicone, an ester, a fragrance, or an antioxidant.

The cosmetic composition may have various forms, including a loose or packed powder, an emulsion having, e.g., a silicone external phase and/or an internal phase that is an aqueous phase, or a gel. The composition may be a lotion or cream.

### Volatile Solvents

Embodiments of the disclosed composition may optionally contain at least one volatile solvent (any non-aqueous medium capable of evaporating on contact with the skin or the lips in less than one hour at room temperature and atmospheric pressure).

Examples of suitable volatile solvents include volatile hydrocarbon-based oils such as, for example, volatile hydrocarbon oils having from 8 to 16 carbon atoms and their mixtures and in particular branched C₈ to C₁₆ alkanes such as C₈ to C₁₆ isoalkanes (also known as isoparaffins), isododecane, isodecane, isohexadecane, and for example, the oils sold under the trade names of Isopar or Permethyl, the C₈ to C₁₆ branched esters such as isohexyl or isodecyl neopentanoate, alcohols, and their mixtures. Preferably, the volatile hydrocarbon-based oils have a flash point of at least 40° C.

Examples of volatile hydrocarbon-based oils include, but are not limited to isododecane, isohexadecane, isodecyl neopentanoate, propylene glycol n-butyl ether, Ethyl 3-ethoxypropionate, Propylene glycol methylether acetate, and an isoparaffin (such as isoparaffin C₁₁-C₁₃ or isoparaffin C₁₁-C₁₂).

The volatile solvent may also be chosen from volatile silicone oils. Optionally, these volatile silicone oils may be linear or cyclic, and may having a viscosity, at room temperature, of less than or equal to 6 cSt. These volatile silicone oils may be selected from silicone oils having from 2 to 7 silicon atoms. These may be substituted or unsubstituted chains. One optional embodiment utilizes silicoe oils having from 2 to 7 silicon atoms and is substituted with alkyl or alkoxy groups of 1 to 10 carbon atoms.

Examples of suitable volatile silicone oils include, but are not limited to, Octyltrimethicone, Hexyltrimethicone, Decamethylcyclopentasiloxane (cyclopentasiloxane or D5), Octamethylcyclotetrasiloxane (cyclotetradimethyl siloxane or D4) Dodecamethylcyclohexasiloxane (D6), Decamethyltetrasiloxane(L4), KF-96 A from Shin Etsu, PDMS (polydimethylsiloxane) DC 200 (1.5 cSt) from Dow Corning, PDMS DC 200 (2 cSt) from Dow Corning, PDMS DC 200 (5 cSt) from Dow Corning, and PDMS DC 200 (3 St) from Dow Corning.

In certain embodiments, the at least one volatile solvent is generally present in the cosmetic composition in an amount ranging from about 5% to about 70% by weight. In certain embodiments, it is present from about 25% to about 50% by weight. In certain embodiments, it is present from about 30% to about 40% by weight.

### Adjuvants

The cosmetic composition may optionally contain an adjuvant, such as a preserving agent, antioxidant, fragrance, sequestrant (such as EDTA), or dyestuff. The amounts of these various adjuvants are those conventionally used in the field under consideration, for example from 0.01% to 10% by weight and preferably from 0.01% to 5% by weight, relative to the total weight of the cosmetic.

The cosmetic compositions may contain at least one cosmetically acceptable colorant such as a pigment or dyestuff. Examples of suitable pigments include, but are not limited to, inorganic pigments, organic pigments, lakes, pearlescent pigments, irridescent or optically variable pigments, and mixtures thereof. A pigment should be understood to mean inorganic or organic, white or colored particles. Said pigments may optionally be surface-treated within the scope of the present invention but are not limited to treatments such as silicones, perfluorinated compounds, lecithin, and amino acids.

Representative examples of inorganic pigments useful in the present invention include those selected from the group consisting of rutile or anatase titanium dioxide, coded in the Color Index under the reference CI 77,891; black, yellow, red and brown iron oxides, coded under references CI 77,499, 77, 492 and, 77,491; manganese violet (CI 77,742); ultramarine blue (CI 77,007); chromium oxide (CI 77,288); chromium hydrate (CI 77,289); and ferric blue (CI 77,510) and mixtures thereof.

Representative examples of organic pigments and lakes useful in the present invention include, but are not limited to, D&C Red No. 19 (CI 45,170), D&C Red No. 9 (CI 15,585), D&C Red No. 21 (CI 45,380), D&C Orange No. 4 (CI 15,510), D&C Orange No. (CI 45,370), D&C Red No. 27 (CI 45,410), D&C Red No. 13 (CI 15,630), D&C Red No. 7 (CI 15,850), D&C Red No. 6 (CI 15,850), D&C Yellow No. 5 (CI 19,140), D&C Red No. 36 (CI 12,085), D&C Orange No. 10 (CI 45,425), D&C Yellow No. 6 (CI 15,985), D&C Red No. 30 (CI 73,360), D&C Red No. 3 (CI 45,430) and the dye or lakes based on cochineal carmine (CI 75,570) and mixtures thereof.

Representative examples of pearlescent pigments useful in the present invention include those selected from the group consisting of the white pearlescent pigments such as mica coated with titanium oxide, mica coated with titanium dioxide, bismuth oxychloride, titanium oxychloride, colored pearlescent pigments such as titanium mica with iron oxides, titanium mica with ferric blue, chromium oxide and the like, titanium mica with an organic pigment of the above-mentioned type as well as those based on bismuth oxychloride and mixtures thereof.

The precise amount and type of colorant employed in embodiments of the disclosed compositions will depend on the color, intensity and use of the cosmetic composition and, as a result, will be determined by those skilled in the art of cosmetic formulation. However, one preferred amount of colorant for use in the present invention is from about 0.5% to about 7.5%, based on the weight of the composition.

These compositions can then be applied to a user's skin or hair using various known application techniques.

### EXAMPLE 1

### Water-In-Silicone

Given a base water-in-silicone formula, nine test formulas were created, utilizing 1% w/w, 2% w/w, and 3% w/w of either Nylon-12, PMMA, or PHB. The base formula was also tested.

### EXAMPLE

| PHASE | CHEMICAL | PERCENT (%) |
|---|---|---|
| A | Dimethicone | 1-20% |
| | Isododecane | 1-20% |
| | Surfactants | 0.1-10% |
| | Filler | 0.1-30% |
| | Silicone MQ Resin | 0.1-15% |
| | Stabilizing Agent | 0.01-2% |
| | Pigments | 0.1-41% |
| B | Water | 5-45% |
| | MAGNESIUM SULFATE | 0.1-2% |
| | Preservatives | 0.01-2% |

To produce, mix ingredients from phase A at room temperature under high shear. Mix ingredients from phase B at room temperature under low shear until homogenous. Slowly add phase B to phase A while mixing to produce the emulsion.

In testing, each formula was applied to the skin. The gloss was measured at 10 minutes, 20 minutes, 30 minutes, 60 minutes, and 24 hours. A 3-mil wet film was prepared on a paper-based substrate and allowed to dry overnight at room temperature. The film was placed and locked in an apparatus containing synthetic sebum, which was exposed to the back side of the film by inverting the apparatus. A gloss meter supplied by BYK (micro-TRI-gloss) was used for the gloss measurements.

At 60 minutes, the gloss for the control was about 8 gloss units, while the other formulations were between 1 and 4. Of the nine test formulas, Nylon was routinely the least glossy formulation, usually having a gloss around 1, while PMMA was the glossiest, rising from around 1 at 10 minutes to about 4 at 60 minutes. The PHB formulas were in between the two, generally rising from about 1 at 10 minutes to about 2 (1%, 2% usage) to 3 (3% usage).

At 24 hours, the gloss for the control was about 28 gloss units, while the test formulas had gloss measurements of between about 3 to about 8 gloss units.

In a multichallenge wear test, a film is deposited onto a surface such as a black byko-chart Black Scrub Panels P121-10N #5015 using a 1 mL drawdown bar. After drying 24 h at 37° C., the deposit can be covered with artificial sebum or sweat. Samples are then abraded using an automatic drawdown machine (Gardco Automatic Drawdown Machine) equipped with Velcro (¾″ white 010 PSA 0172) pieces adhered to the lowest bar. Contact between the Velcro adhered bar and the film interface is made and then the bar is automatically dragged across the sample one time. The film can then be wiped by hand using light force with an absorbent material (for example, bioskin and/or KIMWIPES™ brand delicate task wipers from Kimberly-Clark). Then, it can be determined how much of the sample remains undisrupted on the scrub panel as well as how much of the sample has transferred to the absorbent material using a rating scale such as the one described above to assess the degree of sample removal from the substrate or the degree of sample transferred to the absorbent material, in which 1 is total disruption and extreme transfer to absorbent material, 2 is most disruption with substantial transfer to absorbent material, 3 is half disruption and some transfer to the absorbent material, 4 is minimal disruption and minimal transfer to absorbent material and 5 is essentially no removal and no transfer.

Results are set forth in Table 1 below

| | Sweat | Sweat | Sebum | Sebum | |
|---|---|---|---|---|---|
| | transfer | transfer | transfer | transfer | |
| Formula/ | on | on | on | on | |
| Product | bioskin | Kimwipe | bioskin | Kimwipe | Adhesion |
| 1% Nylon 12 | 5 | 5 | 3 | 3 | 2 |
| 2% Nylon 12 | 5 | 5 | 3 | 3 | 3 |
| 3% Nylon 12 | 5 | 5 | 3 | 3 | 3 |
| 1% PMMA | 5 | 5 | 4 | 3 | 2 |
| 2% PMMA | 5 | 4 | 4 | 3 | 2 |
| 3% PMMA | 5 | 5 | 3 | 2 | 3 |
| 1% PHB | 3 | 5 | 3 | 4 | 3 |
| 2% PHB | 3 | 5 | 4 | 4 | 3 |
| 3% PHB | 3 | 5 | 5 | 4 | 3 |
| Control | 3 | 3 | 3 | 3 | 3 |

### EXAMPLE 2

### Loose Powder Formulation

A loose face powder having the following composition was prepared:

| | | |
|---|---|---|
| PHB | 20-40% | w/w |
| Talc | 15-40% | w/w |
| Mica | 5-20% | w/w |
| Iron oxides | 1-5% | w/w |
| Isocetyl stearate | 1-5% | w/w |
| Preserving agents | <2% | w/w |

The powder spread easily on the face and gave good softness properties.

### EXAMPLE 3

### Packed Powder Formulation

A compact face powder having the following composition was prepared:

| | | |
|---|---|---|
| PHB | 20-40% | w/w |
| Talc | 15-40% | w/w |
| Mica | 5-20% | w/w |
| Iron oxides | 1-5% | w/w |
| Zinc stearate | 1-5% | w/w |
| Bismuth oxychloride | 5-20% | w/w |
| Preserving agents | <2% | w/w |
| Phenyltrimethicone | 1-10% | w/w |
| Glyceryl triisostearate | 1-10% | w/w |

The powder was sieved, poured into a metal dish and then compacted. The powder spread easily on the face and gave good softness properties.

Those skilled in the art will recognize or be able to ascertain using no more than routine experimentation many equivalents to the specific embodiments of the invention described herein. Such equivalents are intended to be encompassed by the following claims.

What is claimed is:

1. A cosmetic composition, comprising:
from 0.1% w/w to 30% w/w of at least one polyhydroxyalkanoate (PHA) in the form of particles having an average diameter (d50) from 0.1 nm to 50 μm,
wherein the composition has a silicone external phase or is in the form of a loose or packed powder.

2. The cosmetic composition according to claim 1, wherein the PHA has a chain length between 1 and 8 carbons.

3. The cosmetic composition according to claim 1, wherein the PHA is selected from the group consisting of: poly-3-hydroxybutyrate (PHB) , poly-3-hydroxyvalerate (PHV) , poly-3-hydroxyhexanoate (PHH), poly-3-hydroxyoctanoate (PHO), poly (3-hydroxybutyrate-co-3-hydroxyvalerate) (PHBV), poly (3-hydroxybutyrate-co-3-hydroxyexanoate) (PHBH) , poly (3-hydroxybutyrate-co-4-hydroxybutyrate) , poly (3-hydroxyoctanoate-co-3-hydroxyundecen-10-enoate) (PHOU), and poly(3-hydroxybutyrate-co-3-hydroxyvalerate-co-4-hydroxyvalerate) (PHBW).

4. The cosmetic composition according to claim 1, wherein the composition further comprises at least one pigment.

5. The cosmetic composition according to claim 1, wherein the PHA is selected to have an oil absorption of between 60 to 150 grams of a desired oil per 100 grams of PHA.

6. The cosmetic composition according to claim 1, wherein the PHA is present in the composition in an amount from 1% w/w to 8% w/w.

7. The cosmetic composition according to claim 1, wherein the PHA is PHB, and wherein the composition has a silicone external phase.

8. A method of using a cosmetic composition, comprising:
providing a composition according to claim 1; and
applying the composition to a user's skin or hair.
